(19) United States
(12) Patent Application Publication
(10) Pub. No.: US 2008/0171951 A1
(43) Pub. Date: Jul. 17, 2008

(54) Integrated System for Collecting, Processing and Transplanting Cell Subsets, Including Adult Stem Cells, for Regenerative Medicine

(76) Inventor: Claude Fell, Nyon (CH)

Correspondence Address: STURM & FIX LLP, 206 SIXTH AVENUE, SUITE 1213, DES MOINES, IA, 50309-4076, US

(21) Appl. No.: 11/908,434

(22) PCT Filed: Mar. 23, 2006
(86) PCT No.: PCT/IB2006/050895

§ 371 Date: Sep. 12, 2007

(30) Foreign Application Priority Data
Mar. 23, 2005 (IB) PCT/IB2005/000747

(51) Int. Cl. A61B 10/02; C12M 3/00

(52) U.S. Cl. 600/573; 604/322; 435/287.2

(57) ABSTRACT

A system for the extraction, collection, processing and transplantation of cell subsets, including adult stem cells and platelets, in particular for tissue repair in regenerative medicine, comprises a set of disposable fluid-transport elements that are pre-connected or that include aseptic connectors for making interconnections between them in an aseptic manner or are adapted to be aseptically connected. The set usually includes three kits of disposable sterile elements, a collection kit, a processing kit, and a transplantation kit packaged in a blister pack on a support such as a tray, having one compartment for receiving each inter-connectable kit of the set. The set includes an extracting device, for example including a needle for bone puncture or vein puncture, for extracting bone marrow or other sources of cell subsets from a patient.

### FIELD OF THE INVENTION

This invention relates to the collection, automated processing and transplantation of cell subsets as found in the bone marrow, peripheral blood, umbilical cord blood or adipose tissue with the objective to locally reinject these cells for repairing tissues. Cell subsets are typically Adult Stem Cells or platelets but more generally include any sub-populations of cells like red blood cells and white blood cells. Such procedures are likely to be performed in a hospital setting or medical facilities having no cell processing laboratory, and that are likely going to be performed by non-specialized technicians. The invention also includes a new type of optical sensor to monitor cell subsets passing through a transparent tube.

### BACKGROUND OF THE INVENTION

Stem cells are defined as cells that have clonogenic and self-renewing capabilities and that differentiate into multiple cell lineages. Whereas embryonic stem cells are derived from mammalian embryos in the blastocyst stage and have the ability to generate any terminally differentiated cell in the body, adult stem cells are part of tissue-specific cells of the postnatal organism into which they are committed to differentiate. Adult stem cells offer practical advantages over embryonic stem cells. Unlike the latter, they do not raise any ethical issue, and can be extracted from the patient himself. They are in abundant supply and are intrinsic to various tissues of the human body. The most accessible sources of adult stem cells are the bone marrow, peripheral blood, umbilical cord blood and possibly adipose tissues, as indicated by recent studies. These cells are capable of maintaining, generating and replacing terminally differentiated cells within their own specific tissue as a consequence of physiologic cell turnover or tissue damage due to injury. Such capability, known as cell plasticity, has led to the development of therapeutic applications targeting the regeneration of defected tissues, with the goal to restore the physiology and functionality of the affected organ. Adult stem cells can give rise to hematopoietic cells as known since many decades, but as found in recent years can also give rise to blood vessels, muscles, bone, cartilage, skin, neurons etc. Such cells are known as mesenchymal stem cells. In addition, platelets prepared as platelets concentrate can be used to accelerate wound healing, and consequently can play a role in regenerative medicine to help in the reconstruction of tissues like bone, skin or other tissues.

Hematopoietic stem cells have been used largely for transplanting patients having undergone chemotherapy in order to restore their hematopoiese. Initially extracted from the bone marrow, they have been sourced more recently from the peripheral blood or umbilical blood, these latter having the highest proliferation capacity. Cells for transplantation require special processing like cell separation, followed sometimes by selection and/or expansion processes. To date, such manipulations have been performed within well-equipped cell-processing laboratories by highly trained personnel that are competent in cell biology and hematology. Such manipulations require labor intensive laboratory preparations involving centrifugation in tubes, density gradient separation, often performed in an open system with the risk of contamination by bacteria, etc.

The new perspectives offered by stem cells in the field of regenerative medicine are challenged by practical issues of manipulating these cells in environments that are unfamiliar with these techniques. One of the main issues is the lack of clean rooms allowing a safe processing of the cells. One can cite as examples the fields of cardiology, orthopedics and neurology that are all experimenting stem cell based therapy, but however lack proper cell processing faculties. Consequently there is a need for simple systems that can process adult stem cells or generally any cell subsets automatically, in a closed system and rapidly in order to provide an on-line cell processing system at the patient's bedside.

### DISCLOSURE OF THE INVENTION

The invention provides a system allowing the extraction, collection, processing and transplantation of cell subsets targeting tissue repair in regenerative medicine. Such system can be offered on a support like a tray that includes individual kits for performing the procedure. The individual kits can be pre-connected or can be equipped with aseptic connectors for making interconnections between them in an aseptic manner, or can be connected using a sterile connecting device like, the SCD from Terumo, operating by welding.

The invention provides a simple system for automatically processing/concentrating cell subsets in a closed system that can provide an on-line cell processing system at the patient's bedside, as set out in claim 1. Embodiments of the invention are set out in the dependent claims.

In one embodiment, the collection container used for harvesting the cell subsets from the patient can be designed in such a way in order to be used as separation chamber as well. Similarly, the receptacle used for collecting the separated cells can be designed in such a way in order to serve as a reinfusion container to deliver the cells back to the patient. The separation of the cells can target a buffy-coat collection or be performed using a density gradient based separation process, followed by a cell washing, based on the system as described in EP-B-912 250 (Claude Fell) and PCT/IB99/020523 (Biosafe). Another way of processing the cells is to use microbeads coated with monoclonal antibodies as described in WO03/009889 (CellGenix/Biosafe).

The combined use of an optical detector which can measure absorption and reflection due to the cells flowing into a transparent tube permits to collect more precisely a particular cell subset like platelets to produce a platelets concentrate. Such platelet concentrate can be obtained in a separate procedure or as a by-product during a procedure targeting a cell subset. The invention also contemplates using the described system for preparing a platelet concentrate for separate use.

The invention thus provides a fully integrated system for bedside intervention that minimizes risks of contamination by using a closed system. It offers a great level of automation and does not rely on any special cell processing expertise. It is suitable for handling any source of cells (such as Adult stem cells, platelets), but particularly for bone marrow stem cell preparation, in an autologous or allogenic setting.

### BRIEF DESCRIPTION OF THE DRAWINGS

The invention will be further described, by way of example, with reference to the accompanying schematic drawings in which:

FIG. 1 is a diagram illustrating the general set-up of a bone marrow processing kit according to the invention;

FIG. 2 shows the symbols used in FIGS. 3-7 to illustrate the different components of the illustrated kits according to the invention;

FIGS. 3A and 3B show two embodiments of a collection kit, one without and one with a filter unit;

FIG. 4 shows a processing kit that can be connected by an aseptic connector to a collection kit as shown in FIG. 3A or FIG. 3B or to a transplantation kit as shown in FIG. 5A or 5B;

FIG. 5A shows individual elements of a transplantation kit and FIG. 5B shows a combination of elements making up a transplantation kit;

FIG. 6 illustrates different combinations of kits for making up a complete system;

FIG. 7 is a diagram of an all-in-one bone marrow processing set in which a rotatable processing chamber constitutes a separation syringe that is used also for collection and transplantation of the cells;

FIGS. 7A, 7B and 7C show the operative configurations of components of the set of FIG. 7 for collection, processing and transplantation, respectively;

FIGS. 8A, 8B and 8C show the principle of the detection of the cells by an optical line sensor using the absorption and reflecting properties of the cells;

FIG. 8D shows a vertical view of the optical line sensor with the location of LED and receiver devices; and

FIG. 9 shows typical output signals of the optical line sensor of FIG. 8.

### DETAILED DESCRIPTION

The invention relates to an integrated system allowing the collection of cell subsets, their processing/concentration and reinfusion of a particular cell subset rich product with the objective of repairing or regenerating an injured or defective tissue.

The process will be described in an autologous setting, meaning that the cells are extracted and reinfused from and to the same patient. Such autologous treatment is generally preferred as there is no immunological reaction or adverse effects due to incompatibilities between donor and receivers. However the principle would remain the same in an allogenic setting.

Stem cells, and more specifically mesenchymal stem cells are found in the bone marrow according to current knowledge, although studies indicate that mesenchymal stem cells exist also in umbilical cord blood, peripheral blood or even in fatty tissues. Although the principle would also apply to these various sources of stem cells, the process described here relates to the processing of bone marrow.

The process consists first of a bone marrow extraction from the pelvic zone, under a local anesthesia. Bone is perforated using a bone marrow extractor for example of the Tyco type. The marrow is aspirated using one or multiple syringes, which are pre-filled with some anticoagulant, usually heparin or a citrate/phosphate solution. A volume of 50 ml is typically collected, but could be a different value. The aspirated bone marrow is generally transferred into a PVC collection bag, either filtrated or not, and can be put on an agitator. The PVC collection bag is then connected, using aseptic techniques, preferably on the system described in EP-B-912 250 and PCT/IB99/02052, and separation and concentration of stem cells is then performed accordingly. Other centrifugal processing chambers can be used (e.g. where the rotation axis is not parallel to the axis of a cylindrical processing chamber) or using flexible containers.

EP-B-0 912 250 (C.FELL), the contents whereof are herein incorporated by way of reference, describes a system for the processing and separation of biological fluids into components, comprising a set of containers for receiving the biological fluid to be separated and the separated components, and optionally one or more additional containers for additive solutions. A hollow centrifuge processing chamber is rotatable about an axis of rotation by engagement of the processing chamber with a rotary drive unit. The processing chamber has an axial inlet/outlet for biological fluid to be processed and for processed components of the biological fluid. This inlet/outlet leads into a separation space of variable volume wherein the entire centrifugal processing of biological fluid takes place. The processing chamber comprises a generally cylindrical wall extending from an end wall of the processing chamber, this generally cylindrical wall defining therein the hollow processing chamber which occupies a hollow open cylindrical space coaxial with the axis of rotation, the axial inlet/outlet being provided in said end wall coaxial with the generally cylindrical wall to open into the hollow processing chamber. The processing chamber contains within the generally cylindrical wall an axially movable member such as a piston. The separation space of variable volume is defined in an upper part of the processing chamber by the generally cylindrical wall and by the axially movable member contained in the generally cylindrical wall of the processing chamber, wherein axial movement of the movable member varies the volume of the separation space, the movable member being axially movable within the processing chamber to intake a selected quantity of biological fluid to be processed into the separation space via the inlet before or during centrifugal processing and to express processed biological fluid components from the separation space via the outlet during or after centrifugal processing. Means are provided for monitoring the position of the movable member to thereby control the amount of intaken biological fluid and the expression of separated components. The system further comprises a distribution valve arrangement for establishing selective communication between the processing chamber and selected containers or for placing the processing chamber and containers out of communication.

According to PCT/IB99/02052, such a system is arranged to operate in a separation and in a non-separation transfer mode, which provides greater possibilities for use of the system including new applications which were previously not contemplated, such as separation of hematopoietic stem cells and in general laboratory processing. Thus, the system can be arranged to operate such that:
in the separation mode fluids can be intaken into the processing chamber while the chamber is rotating or stationary, fluid intaken into the chamber is centrifuged and separated into components, and the separated components expressed while the chamber is rotating or, optionally, for the last separated component, while the chamber is stationary; and
in the transfer mode the processing chamber intakes fluid and expresses fluid with the chamber stationary. The valve actuation arrangement is actuable to transfer amounts of fluid from one container to another via the processing chamber, by moving the member, without centrifugation or separation of the fluid into components, and the means for monitoring the position of the movable member controls the amounts of non-separated fluids transferred.

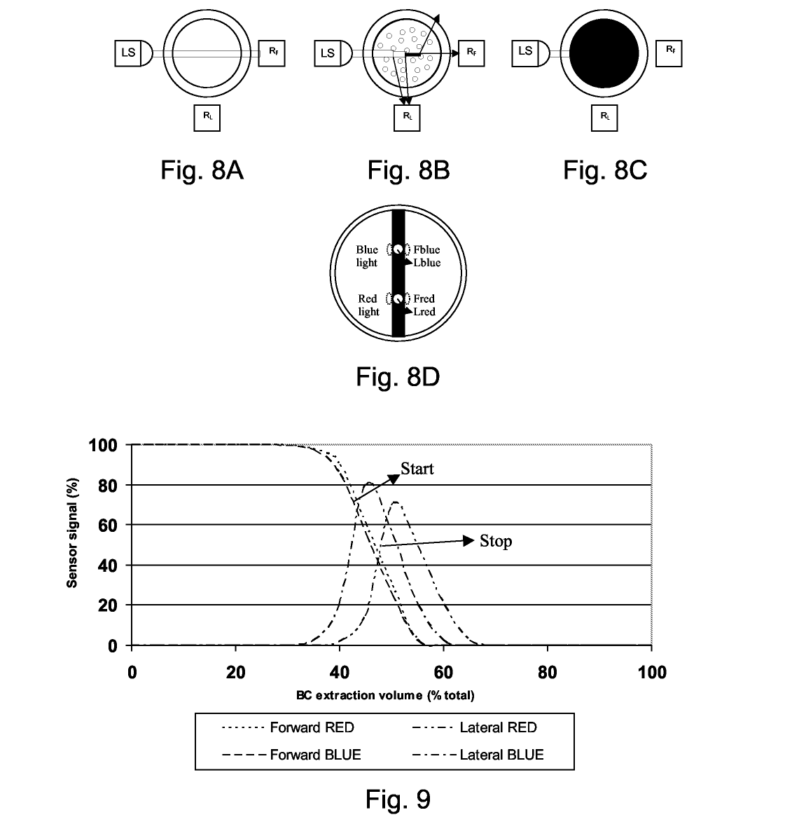

In the new application according to the present invention, which advantageously uses the separation chamber according to EP-B-912 250 and PCT/TB99/02052, separation can target a buffy-coat collection that allows the highest recovery in stem cells without any specific cell subset targeting. The initial product, which sources are those described above, is introduced into the separation chamber by lowering the piston. Once the product has been loaded into the separation chamber—this is detected by the optical line sensor placed near the entry of the separation chamber—a sedimentation cycle of typically 5-10 minutes, produces a buffy-coat layer between the plasma and the red blood cells layer. At the extraction, the plasma is extracted first by moving the piston up. An optical line sensor, for example that described with reference to FIG. 8, detects the cells which belong to the buffy-coat and adapts the different parameters (extraction speed, extracted volume, centrifugation speed) to optimize the cell recovery, depending on the desired volume and the time to process constraints. The buffy-coat cells are extracted to the dedicated bag or vial (depending the configuration of the processing kit). The remaining red blood cells are either extracted to a dedicated bag or kept in the chamber (to save process time). Depending on parameters optimized during the buffy-coat extraction phase, a successive sedimentation/extraction cycle(s) as the one described above can restart. This will complete the buffy-coat extracted volume optimizing its characteristics depending on the final application of the cellular product.

To select a more specific cell subpopulation, the principle described above (filling/sedimentation/extraction) can be used by the same type of optical line sensor which detects by reflection and absorption a more specific cell subset. During the plasma extraction, the absorption and reflection of the liquid are very low. When cells begin to flow in the tube, both absorption and reflection of the effluent product increases. The reflection is also dependant from the type and size of the cells. This dependence permits the selection of a particular cell subset. Finally when the cell concentration is high, the absorption is at the maximum level and reflection is not possible anymore. This can be used to detect cell subsets which have different sizes like platelets and create a platelet concentrate.

To obtain a cell subset selection, a preferred method is to use a density gradient media which targets more specifically a defined cell subpopulation. This will increases the purity of the product, by reducing the contamination in red blood cells and other not wanted cell subsets. The density gradient media is chosen according to the targeted subset. For example, to target mononuclear subset, a Ficoll™ based media can be used. In such case, density gradient media is first introduced in the separation chamber. Bone marrow is then slowly introduced by lowering the piston, typically at a rate of 5 ml/min, in order to deposit the cells on the layer of density gradient media. Red cells and granulocytes will tend to go through the layer of density gradient media, while mononuclear cells and platelets will remain in front of the layer. When the entire volume of bone marrow is introduced, as detected by an optical line sensor (FIG. 8) on the top of the machine, the piston is stopped and a sedimentation step of, for example, 10-20 min is initiated. An additional dilution could be automatically performed by the system after the complete aspiration of the product, thanks to an isotonic solution connected to the system. Centrifuge speed can be increased to reduce this sedimentation time. Collection then starts by moving the piston up. The liquid supernatant contains only plasma. The first cells then follow, causing the effluent tubing to become opaque, as detected by the optical line sensor. This triggers the collection of the mononuclear cells, which encompass the targeted stem cells. After a volume predefined by the operator menu, or when the effluent line clears again, cell collection stops, and the remaining content of the separation chamber volume is collected in a waste bag until the chamber is completely empty. At this stage, the separation chamber is rinsed from all residual red blood cells thanks to an isotonic solution. The collected cells are reintroduced in the separation chamber, followed, or preceded, by a washing solution like saline/albumine solution (alternatives with phosphate buffered solution or other can be also used). The cells and the washing solution are mixed. The piston will stop after a predetermined volume or when the chamber is completely full. A new sedimentation step is then performed, during which, the collection bag can be washed using the supernatant produced during sedimentation to remove traces of density gradient media. The supernatant (consisting of the washing solution and density gradient media) is then expressed. The process is stopped when the first cells appear again in the effluent line, or can be repeated to obtain a better washing. Cells are finally collected into a collection container that can be specially designed to facilitate further use of the collected cells. When needed the chamber is rinsed. Such cells are readily available for reintroduction into the targeted organ of the patient, or can be further manipulated for selection or expansion purposes. To this effect, the system could re-suspend the cells directly in the desired culture medium.

A more refined separation than using density gradient media consists in incubating the bone marrow in a medium that contains microbeads coated with monoclonal antibodies. Such separation method is described in publication WO03/009889 (CellGenix/Biosafe). The procedure is then as follows. A product containing microbeads linked to a specific antibody is mixed to the blood product containing the cells of interest. After some incubation time, the microbeads will adhere to the surface of the targeted cells, causing their density to change. The mixture is then poured into the separation chamber and a buoyant density separation is initiated as described in earlier patents. When the sedimentation is complete, the supernatant is extracted from the chamber into the waste bag, and then the red cells are also discarded. The cells of interest, marked with the microbeads and therefore having the highest density, will be the last ones to exit the chamber. They can be collected in an appropriate container and, if needed, subsequently washed to remove the antibody solution.

In case of immediate reintroduction after processing has been performed, the collected cells can be connected, using aseptic techniques, to the device allowing their transplantation to the patient. For cardiology applications, such device can be a balloon catheter as used in angiography to locally reinject these cells, like for instance in association with acute myocardial infarction treatments. In this case, a quantity of 10 ml of the concentrated stem cells in steps of 3 ml is reinjected, inflating the balloon at regular interval, allowing the spreading of the stem cells. Such method has been described by Zeiher et al in a scientific paper (TOPCARE—Circulation October 2002).

In case of a platelet concentrate collection, harvested platelets can be used alone or in combination with thrombine possibly obtained as well from the patient's plasma, to form a platelet gel that will facilitate wound healing. Such platelet gel contains growth factors that can advantageously stimulate tissue repair either alone or in association with stem cells.

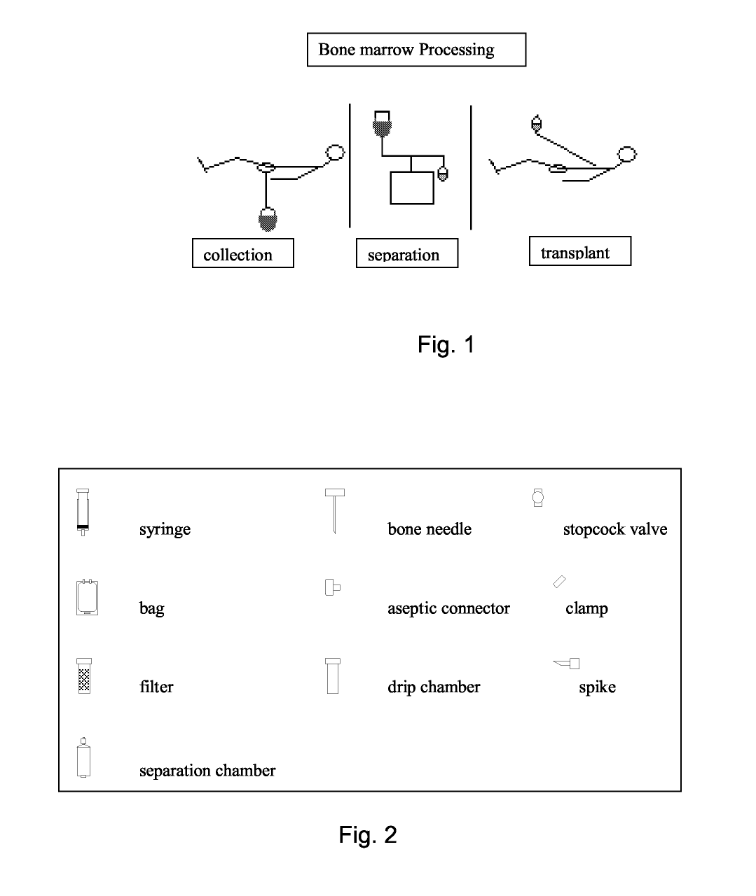

The whole process can be performed at the patient's bedside, and is therefore considered as an on-line process, as illustrated schematically in FIG. 1. This provides significant advantages, in safety, logistic and response time, and does not rely on any specific expertise of cell processing.

The collection of various targeted cell subsets can be done during the same collection procedure but with the objective to use such cell subsets at different time intervals during the same operation.

The invention provides a system or “custom pack” that contains already the individual disposable sterile sets for performing the collection, separation and transplantation respectively. Such pack can be presented in a “blister” having 3 compartments each containing a disposable set or kit: one set for the bone marrow extraction, one set for the bone marrow separation, preferably of a type based on the system described in EP-B-912 250 and PCT/IB99/020523 and one set for the reinjection of cells. Each set can have some variations, the one having the highest versatility being the transplantation set, as it depends of the targeted tissue to treat (eg. bone, muscle, vessel, etc). Individual set configurations are illustrated in FIGS. 2-7.

Possibly these sets can be preconnected altogether or two of the three can be preconnected, if for instance one wants to use a fully closed system. If not preconnected, a practical solution consists of using specially designed aseptic connectors, like the Medlock system offered by PALL (ref. ACD) and described in U.S. Pat. Nos. 3,650,093, 5,868,433, 6,536,805 and 6,655,655, to ensure that connections are performed under aseptic connections, thus maintaining the criteria of a closed system. Another possibility would be to connect the set using a sterile connecting device. Any of the above configurations—pre-connected, or connected with an aseptic connecting device or a sterile connecting device—will provide a functionally closed system. Such functionally closed systems eliminate the need of clean rooms or laminar flow systems, a very important advantage in operating room or interventional unit environments, which generally are not equipped to meet these requirements.

Another refinement of the invention consists in a bone marrow aspiration container that will act as the separation chamber in second stage, referred as a processing chamber. Its design is similar to the separation chamber as described in PCT/IB99/020523, and it can be fitted with a special needle for perforating the pelvic bone. It is prefilled with anticoagulant or can be primed with anticoagulant prior starting the collection. Once the bone has been perforated, bone marrow is aspirated by moving down the piston of the processing chamber, activated by a manual or electrical vacuum source. The processing chamber is then inserted into the centrifuge of the machine, and a set consisting of an array of tubing lines and bags is connected on the chamber. Separation can then be initiated according to the process described above, using for instance a buffy-coat centrifugation protocol. Another refinement of the invention consists in collecting the separated cells into a special container that can easily be connected or fitted to the system reinfusing the cells back to the patient. Such container can be a graduated syringe fitted with a Y connector having one end connected to the separation set, and the other end equipped with a luer lock connector for subsequent connection to a catheter.

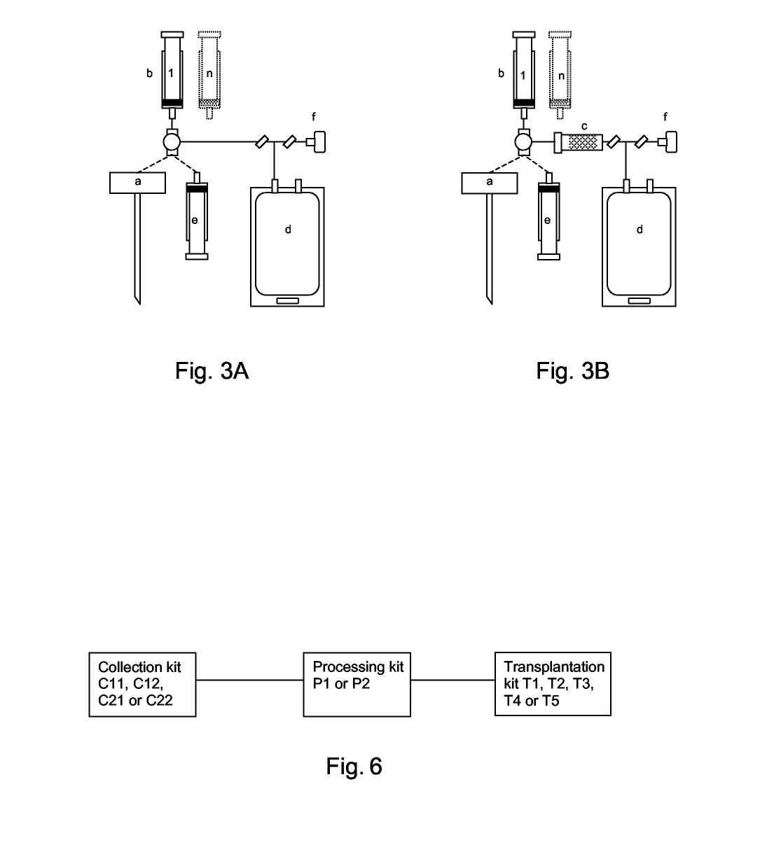

FIG. 3A shows a collection kit without filter and FIG. 3B with filter. The collection kit incorporates everything necessary to perform the marrow aspiration:
a. The input point will for example be a bone marrow extractor (e.g. of the TYCO type) and typically includes a needle for bone puncture or a needle for vein puncture. It can be directly connected to the rest of the kit via a stopcock valve or other valve.
b. One or more syringes (1 . . . n) as needed to perform the aspiration.
c. A filter can be inserted on the collection kit to filter the marrow after collection, as illustrated in FIG. 3B.
d. The collected marrow (filtered or unfiltered) is then stored into a transfer bag prior processing.
e. An additional syringe can be used to add a dilution product to the marrow and/or rinse the filter if applicable.
f An aseptic connector (Pall ACD or similar) can be used
The collection kit can for example be configured as follows:
C11 Collection kit without filter c with bone needle a pre-connected on the processing kit. Quantity X of syringes b.
C12 Collection kit without filter with bone needle a not pre-connected on the processing kit. Quantity X of syringes b.
C21 Collection kit with filter c with bone needle a pre-connected on the processing kit. Quantity X of syringes b.
C22 Collection kit with filter c with bone needle a not pre-connected on the processing kit. Quantity X of syringes b.

In all these configurations the bone needle can be replaced with a needle for vein puncture. FIG. 4 shows a processing kit that is adapted to be connected to the previously-described collection kit via an aseptic connection.
a. The aseptic connection could be performed via an aseptic connector (such as Pall ACD or others) or a spike connector under aseptic conditions.
b. An optional drip chamber could be used to prevent bubbles entering into the processing path.
c. The direction of the fluid path can be chosen by a stopcock valve ramp mounting three stopcocks in this example, or another type of valve e.g. a multiport valve.
d. A separation chamber for example as described in EP-B-912 250 and PCT/IB99/02052 is used for the separation process.
e. Additional product(s) (isotonic solution, culture medium, . . . ) is/are entered by a line equipped with one or more connections (spike connectors, . . . −1 . . . n).
f A satellite or waste bag is used for discarded product and density gradient media input.
g. An output line with an optional intermediate bag guides the product to the transplant kit.
h. An aseptic connector (Pall ACD or similar) could be used on this line.
The main variants of the processing kit include:
P1 processing kit without intermediate bag
P2 processing kit with intermediate bag.

FIG. 5A shows possible individual elements of a transplantation kit and FIG. 5B shows one possible combination of elements making up a transplant kit. The transplantation kit is adapted to be connected to the above described processing kit via an aseptic connection and will contain final product for transplant. The aseptic connection could be performed via an aseptic connector (such as Pall ACD or others) or a spike connector under aseptic conditions.

The transplantation kit could include:
T1 a bag
T2 a collection vial
T3 a syringe
T4 a specific device for transplant (e.g. a catheter for myocardial infarction)
T5 a combination of T1-T4.

Generally speaking, the transplantation kit will as a minimum include at least one specific device for transplant T4 which can be combined with various combinations of the other components for example a bag T1 or a collection vial T2, and/or a syringe T3.

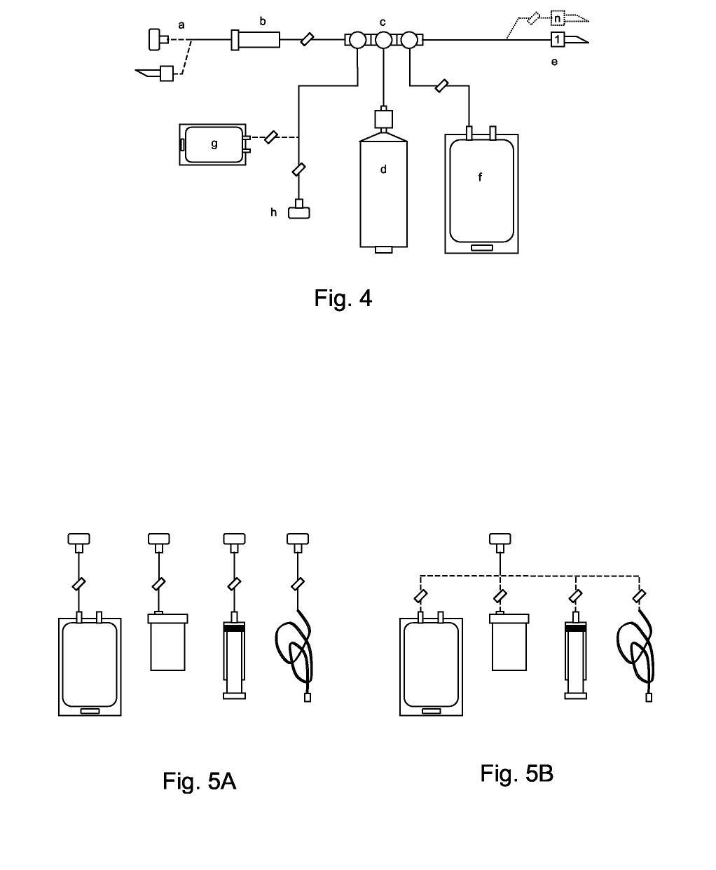

FIG. 6 illustrates different combinations for making up a complete system. The complete system can for example be composed of any combination of a collection kit (C11 to C22), a processing kit (P1 or P2) and a transplant kit (T1-T4), as described above.

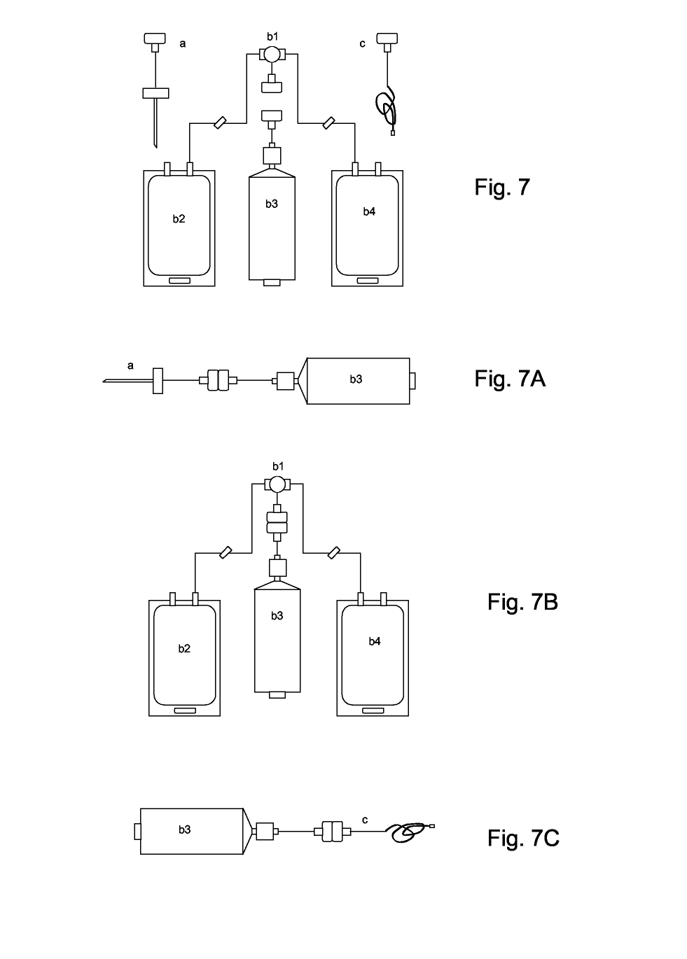

FIG. 7 is a diagram of an all-in-one bone marrow processing set in which a rotatable processing chamber b3 for example as described in EP-B-912 250 and PCT/IB99/020523 constitutes a separation syringe that is used also for collection and transplantation of the cells. The collection kit a consists of the input point, for example a bone marrow extractor (e.g. of the TYCO type) fitted with an aseptic connector for connection to the processing chamber b3. The processing kit b comprises a stopcock valve b1 connected to a washing bag b2 and to a density gradient media/waste bag b4, as well as the separating/processing/transplant chamber b3 that is connectable by an aseptic connector to the stopcock b1, or to the collection kit a, or to the transplantation kit c. The transplantation kit c consists of a specific device for transplant (e.g. a catheter for myocardial infarction) fitted with an aseptic connector for connection to the processing chamber b3.

FIGS. 7A, 7B and 7C show the operative configurations of the components of the set of FIG. 7 for collection, processing and transplantation, respectively.

In FIG. 7A, the collection/processing chamber b3 is connected by an aseptic connector to the input point of the collection system a, so that the processing chamber serves for the collection of the extracted stem cells. Intake of the stem cells is controlled by displacement of the processing chamber b3's piston.

In FIG. 7B, the collection/processing chamber b3 is connected by its aseptic connector to the stopcock b1 that connects it selectively to the washing bag b2 and to the density gradient media/waste bag b4 for the above-described processing operations, which terminate with the processed/concentrated stem cells being returned to the processing chamber b3. Thereafter the processing chamber b3 serves as reinfusion chamber.

FIG. 7C shows the processing chamber b3, after disconnection from the stopcock valve b1 of the processing kit, connected to the transplant device of the transplantation kit c by an aseptic connector. In this configuration, reinfusion of the processed stem cells into the patient can be controlled by displacement of the processing/reinfusion chamber b3's piston.

This embodiment relies on the use of the aseptic connectors to connect the processing chamber b3 selectively to the collection kit a, or to the remainder of the processing kit via the stopcock valve b1, or to the transplantation kit c for carrying out the sequential collection, processing and reinfusion operations. This provides a particularly compact system that does not include any non-used elements and is convenient to use.

FIGS. 8A, 8B and 8C show the principle of the detection of the cells by an optical line sensor LS using the absorption and reflecting properties of the cells through a transparent tube. FIG. 8A is the configuration of a tube containing clear liquid, where light from LS is unreflected and passes directly to a forward detector R on the light axis. FIG. 8B is the configuration of a tube containing cells in suspension in a clear liquid; in this case the cells reflect light in random directions and is captured both by the forward detector R and by a lateral detector R disposed at about 90° to the axis. FIG. 8C is the configuration of a tube containing opaque liquid where no light is reflected. FIG. 8D shows a vertical view of the optical line sensor with the location of LED and receiver devices, in particular showing the positions of the Forward Blue (Fblue), Lateral Blue (Lblue), Forward Red (Fred) and Lateral Red (Lred) light.

FIG. 9 shows the typical signals of the optical line sensor, that are recorded from the “forward” and “lateral” sensors. The information obtained from the “lateral” reflected signals can be used as triggers for starting or ending the collection. The sensor output value (Y axis) is the buffy-coat (BC) extraction volume in percentage of the maximum level. The X axis contains the information of the volume passing through the tube (also in percentage of the total volume).

It is to be understood that this invention may be embodied in several different forms without departing from its spirit or essential characteristics. The scope of the invention is defined in the appended claims, rather than in the specific description preceding them. All embodiments that fall within the meaning and range of equivalency of the claims are therefore intended to be embraced by the claims.

1. A system for the extraction, collection, processing and transplantation of cell subsets, including adult stem cells and platelets, in particular for organ repair in regenerative medicine, the system comprising a set of disposable sterile fluid-transport elements that are pre-connected or that include aseptic connectors or are adapted for making interconnections between them in an aseptic manner, the set including:
an extracting device, for example including a needle for bone or vein puncture, for extracting bone marrow or other sources of cell subsets from a patient;
at least one chamber for the collection, processing and reinfusion of the cell subsets extracted from the patient, including a collection chamber pre-connected or connectable to the extracting device for harvesting the cells extracted from the patient by the extracting device; a processing chamber adapted to cooperate with processing equipment to perform processing and transfer operations on the harvested cells; and a reinfusion chamber for storing processed cells to be delivered back to the patient; wherein the collection, processing and reinfusion chambers are separate and are pre-connected or interconnectable, or wherein a multi-purpose processing chamber provides the combined functions of a collection-processing chamber, a processing-reinfusion chamber or a collection-processing-reinfusion chamber; and
a transplantation device pre-connected or connectable to the reinfusion chamber for delivering processed cells back to the patient.

2. The system of claim 1, wherein the set of disposable elements is packaged in a blister pack on a support such as a tray, the blister pack having one compartment for receiving the entire interconnected set or a plurality of compartments each receiving a part of the set that includes an aseptic connector for connection to another part of the set.

3. The system of claim 1, wherein the set of disposable elements comprises three kits of disposable elements, a collection kit, a processing kit, and a transplantation kit.

4. The system of claim 3, wherein the collection kit comprises a bone marrow extractor device alone or in combination with: at least one syringe; a transfer bag forming the collection chamber, and optionally an aseptic connector for connection to the processing kit.

5. The system of claim 4, wherein the collection kit further comprises a filter connected or connectable between a syringe and a transfer bag.

6. The system of claim 3, wherein the processing kit comprises a processing chamber and at least one disposable container connected to the processing chamber via at least one stopcock valve or a multiport valve allowing selective transfer of fluids to and from the processing chamber and to and/or from the disposable container(s), the processing chamber being connected to the collection kit or having an aseptic connector for connection to the collection kit or being adapted for making an aseptic connection, and the processing chamber also being connected to the transplantation kit or having an aseptic connector for connection to the transplantation kit or being adapted for making an aseptic connection.

7. The system of claim 6, wherein the processing kit further comprises a line equipped with one or more connectors for connecting additional containers to the stopcock valve or multiport valve.

8. The system of claim 3, wherein the transplantation kit comprises at least one transplantation device or a combination of at least one transplantation device with at least one of: a collection bag; a collection vial; and a syringe.

9. The system of claim 1, wherein the processing chamber is a hollow centrifugal processing chamber having an inlet/outlet for the cells to be processed and for the processed cells, the processing chamber containing a movable member which defines a separation space of variable size for receiving the cells, the member being movable to intake a selected quantity cells to be processed into the separation chamber via said inlet and to express processed cells from the separation chamber via said outlet.

10. The system of claim 9, wherein the centrifugal processing chamber is generally cylindrical and is rotatable about the cylinder axis, and the movable member is a piston fluid-tightly movably mounted in the centrifugal processing chamber.

11. The system of claim 1, which comprises:
a device for extracting bone marrow or other sources of cell subsets from a patient, said device being connectable by an aseptic connection to the processing chamber for collecting stem cells extracted by the device in the processing chamber;
at least one disposable container connected to the processing chamber via at least one stopcock valve or a multiport valve allowing selective transfer of fluids to and from the processing chamber and to and/or from the disposable container(s), the processing chamber being connectable to the stopcock or multiport valve via an aseptic connector; and
at least one transplantation device connectable to the processing chamber by an aseptic connection, for the processing chamber to act as reinfusion chamber for the delivery of processed cells therein to the patient.

12. The system of claim 1, wherein the processing chamber is arranged to produce a particular cell subset enriched product (including Adult Stem Cells and platelets).

13. The system of claim 1, wherein the processing chamber is arranged to separate the stem cells using a density-gradient based process followed by cell washing.

14. The system of claim 1, arranged to process the stem cells using microbeads coated with monoclonal antibodies.

15. The system according to claim 1, comprising an optical line sensor for detecting differential reflection of light by a cell subset passing through a transparent tubing.

16. A method of using the system according to claim 1 to prepare a platelet concentrate for separate use.

17. A method of collecting and processing a cell subset extracted from a human being for transplantation into the human being in particular for organ repair in regenerative medicine, comprising collecting the extracted cells in a collection chamber connected to an extraction device through which the cells in particular bone marrow containing stem cells are extracted, processing the cells in a centrifugal processing chamber which is the same as the collection chamber or which is connected to the collection chamber, and collecting the processed cells in a reinfusion chamber which is the same as the processing chamber or is connected to the processing chamber, the reinfusion chamber being connected to a transplantation device connected to the reinfusion chamber for delivering processed cell subset(s) back to the patient.

18. The method of claim 17 which is carried out using a system as claimed claim 1.

19. An optical sensor which is able to measure absorption and reflection of a cell subset passing through a transparent tubing.
